(19) United States
(12) Patent Application Publication
(10) Pub. No.: US 2013/0105371 A1
(43) Pub. Date: May 2, 2013

(54) DETECTION DEVICE AND METHOD

(71) Applicants: Aleksandr Frorip, Elva (EE); Christoph Nacke, Tartu (EE)

(72) Inventors: Aleksandr Frorip, Elva (EE); Christoph Nacke, Tartu (EE)

(73) Assignee: B. BRAUN AVITUM AG, Melsungen (DE)

(21) Appl. No.: 13/702,828

(22) PCT Filed: Jun. 10, 2011
(86) PCT No.: PCT/EP2011/059651

§ 371 Date: Jan. 11, 2013

(30) Foreign Application Priority Data
Jun. 11, 2010 (DE) 10 2010 023 486.9

(51) Int. Cl. A61M 1/14; G01N 21/64

(52) U.S. Cl. CPC A61M 1/14; G01N 21/64
USPC 210/93; 250/458.1; 250/461.1; 356/72; 250/459.1; 210/96.2

(57) ABSTRACT

Detection device for quantitative detection of predetermined molecules, in particular medium size protein molecules, in a sample liquid with a light source for emitting measurement radiation, a section for irradiating the sample liquid and a light detector for measuring the detection radiation exiting the section for irradiating the sample liquid, as well as an analysis stage located downstream of the light detector for analyzing the detector signal, wherein the light source is designed such that the measurement radiation has a spectral range exciting the intrinsic fluorescence of a molecule to be determined, and the light detector is designed such that at least one spectral range of the detection radiation is measured, in which the molecule emits intrinsic fluorescence radiation.

### CROSS REFERENCE TO RELATED APPLICATIONS

This application is the U.S. national phase application of PCT International Application No. PCT/EP2011/059651 filed Jun. 10, 2011, which claims priority to German Patent Application No. DE 10 2010 023 486.9 filed Jun. 11, 2010, the contents of each being incorporated by reference in their entirety.

### FIELD OF THE INVENTION

The invention relates to a detection device for quantitative detection of predetermined molecules, in particular medium size protein molecules, in a sample liquid with a light source for emitting measurement radiation, a section for irradiating the sample liquid and a light detector for measuring the detection radiation exiting the section for irradiating the sample liquid, as well as an analysis stage located downstream of the light detector for analyzing the detector signal. It relates further a corresponding method for detection and finally a use of the method as well as a dialysis apparatus.

### BACKGROUND OF THE INVENTION

By means of dialysis treatment the body of a human (or of an animal) with reduced or lost renal function is supported in the removal of waste products of the metabolism from the blood, up to entire takeover of the renal function by the dialysis apparatus. Already years ago it was determined desirable controlling a dialysis treatment in dependence of its success—thus the time-dependent level of blood purification—in order to on the one hand allow a sufficient effect of this complicated treatment which is burdensome for the patient, and on the other hand to avoid the expense of unnecessary treatment time. There have been developed model approaches for assessing the success of treatment during treatment and have been found measurement parameters, whose current value is sufficiently representative for the success of treatment and which can be measured during a treatment.

Among others the urea content can be measured in the spent dialysis liquid exiting the dialysis apparatus, and can be used as a measure for the success of treatment. However, this requires taking a sample and laboratory analysis, and therefore is hardly applicable “online” during the treatment. Besides there was developed a control method upon use of conductibility sensors which measure enzymatically induced changes in conductibility of exiting dialysis liquid which are caused by hydrolysis of urea (or other key molecules) in the dialysis liquid. During use of this method however practical problems occurred during the calibration of the sensors and the balancing of a (case-dependent varying) base conductibility.

In WO 99/62574 is thus proposed a method for determination of the content of waste products in the dialysis liquid during a dialysis treatment which uses a spectral photometric analysis. In WO 2008/000433 A1 are also proposed a spectroscopic detector and a method with similar application which are specifically intended for the detection of blood and biologic marker compounds in liquids—such as also in dialysis liquid. A corresponding blood detector serves in dialysis apparatuses in particular for the fast measurement of patient critical conditions caused by operational malfunction of the device (as for example a membrane rupture of the membrane filter, interchanging of inlets or haemolysis). The latter document also contains several references for further state of the art.

### SUMMARY OF THE INVENTION

Aspects of the present invention provide an improved detection device and method for detection of said manner which operate specifically of high degree and reliably, and are beneficially applicable for monitoring and eventually controlling of dialysis treatments.

The invention is based on using the intrinsic fluorescence radiation of certain molecules which are regularly contained in a liquid to be examined and whose content is to be determined for the quantitative detection. It further includes the idea of operating the light source thereto such that the measurement radiation has a spectral range exciting the intrinsic fluorescence of a molecule to be determined, and the light detector is designed such that at least one spectral range of the detection radiation is measured, in which the molecule emits intrinsic fluorescence radiation.

Thereby a possibility for detection of molecular mixtures, especially of medium-sized molecules such as beta-2-microglobin, albumin, etc. is established, whose absorption bands are strongly overlapped, and therefore can not be sufficiently specifically detected by absorption measurements. The use of additional markers becomes superfluous so that the implementation is simplified, and it is a continuous detection method and thus the ongoing (“Online”) monitoring of operating conditions or methods is possible in which the content of said molecules in a liquid is significant for the process status.

In an advantageous embodiment it is intended that the light source is a UV-radiation source emitting detection radiation exciting the intrinsic fluorescence of tryptophan, in particular at 280 nm, and the light detector is a photodiode or a photomultiplier, which is sensitive in a spectral range, in which intrinsic fluorescence radiation of tryptophan is emitted, in particular above 340 nm. In particular, a narrowband filter is connected ahead of the light detector, which particularly defines a part of the spectral range between 340 nm and 400 nm as a detection range.

For the measurement of the intrinsic fluorescence radiation and its separation from the measurement radiation (changed by absorption effects) it is intended in advantageous manner that the light detector has a direction of arrival of light which is inclined with respect to a direction of emission of the light source.

A further embodiment is designed that an additional light detector for detecting the effect of the absorption of the sample liquid relating to section for irradiating the sample liquid is arranged to the light source such that its direction of arrival of light coincides with the direction of emission of the light source. It is hereby thus combined a spectral photometric analysis of absorptions effects with the analysis of the intrinsic fluorescence radiation which enables additional possibilities for information and eventually a precise measurement of various molecules (such as protein molecules, specifically metabolism waste products) in the liquid to be examined.

In a further advantageous embodiment the detection device is set up for continuous operation and then particularly applicable for online monitoring of dialysis treatments.

In the proposed dialysis apparatus the section for irradiating the sample liquid is designed in the form of a section of a measurement cell or a liquid conduit which is passed through by spent dialysis fluid as the sample liquid being conveyed from the dialysis apparatus. In an embodiment of such a device a display unit which is connected with the analysis stage of the detection device is provided for displaying the analysis result or data derived from it and/or a storage device for storage of the detection result or data derived from it and/or a transmission device for transmission of the detection result or data derived from it to an external processing unit.

In a further advantageous embodiment the analysis stage of the detection device is connected with an input of a control device of the dialysis apparatus such that the operation of the dialysis apparatus is controllable in dependence on the detection result of the detection device.

Advantageous embodiments of the proposed method as well as its application for monitoring a dialysis treatment mainly arise from the above described apparatus aspects and are therefore not mentioned here again.

### BRIEF DESCRIPTION OF THE DRAWINGS

The invention is best understood from the following detailed description when read in connection with the accompanying drawings. Included in the drawings are the following figures:

FIG. 1 is a schematic drawing of an embodiment of the inventive detection device,

FIG. 2 is a schematic drawing of a dialysis apparatus in which an inventive detection device is connected, and

FIGS. 3A and 3B are drawings for explanation of the functionality of the invention during application.

### DETAILED DESCRIPTION OF THE PREFERRED EMBODIMENTS

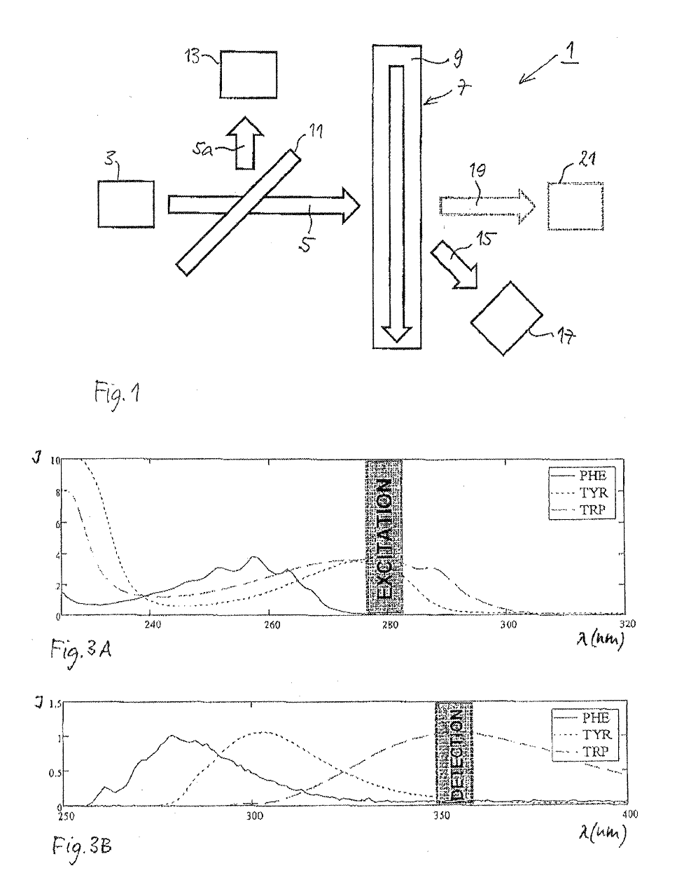

FIG. 1 shows schematically a detection device 1 according to one embodiment of the invention in which a UV-LED 3 (at other position also referred to as light source) emits a measurement radiation 5 on a section for irradiating the sample liquid 7 in which a sample liquid 9 resides. By a semi-permeable mirror 11 one part 5a of the detection radiation is deflected to a first detector (in the following also referred to as “light detector”) 13 for establishing a reference channel. The detection radiation 5 passing through the semi-permeable mirror 11 is effective on certain molecules being contained in the sample liquid 9 as excitation radiation for creation of intrinsic fluorescence radiation 15. This radiation reaches the second detector 17 whose direction of arrival of light is inclined with respect to the direction of emission of the detection radiation 5, and which thus does not measure any radiation passing through the section for irradiating the sample liquid. A part of the radiation 19 passing through in the direction of the detection radiation however is measured by a third detector 21. With this (detector) an absorption measurement of the detection radiation is thus carried out. Details of the analysis of the fluorescence and absorption signals, especially upon regarding those signals gathered in the reference channel for calibration, are not described here since they are common knowledge to the skilled person.

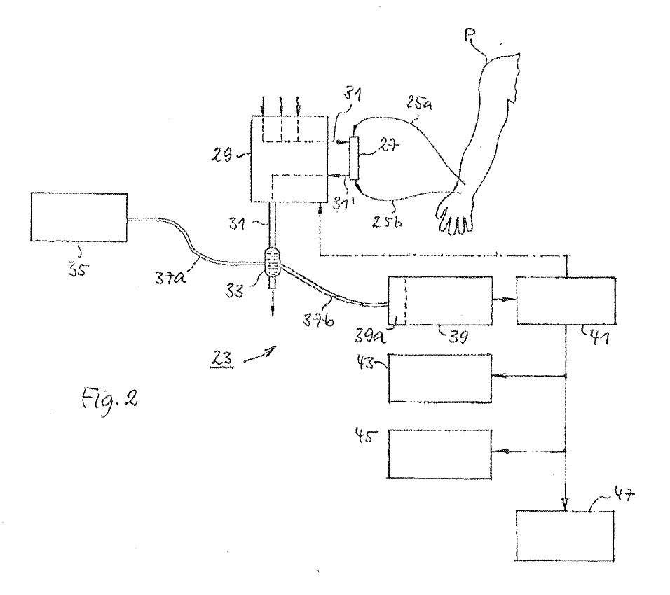

FIG. 2 shows in the form of a block diagram a dialysis apparatus 23 which is provided with an inventive detection device for monitoring and controlling a treatment. Via connection hoses 25a, 25b blood of a patient P is fed to a dialysis cell 27 or conveyed back to the body. From several (here not further specified) components a dialysis liquid 31 is prepared in a dialysis apparatus 29 which is fed to the dialysis cell 27 and conveyed away from it as spent dialysis liquid 31′. In the dialysis cell 27 it has absorbed waste products from the body of patient P via a there provided dialysis membrane, contains thus certain protein molecules whose content is a measure for the status of the dialysis treatment.

Via an outlet conduit 31 of the dialysis apparatus 29 the spent dialysis liquid is passed through the measurement cell 33 and after passage it is discarded. (UV-)measurement radiation generated by light source 35 reaches the measurement cell via a first glass fiber conduit 37a, and via a second glass fiber conduit 37b attached to the opposite measurement cell wall which is inclined to connection direction of the first glass fiber conduit 37a the detection radiation (intrinsic fluorescence radiation) reaches to a detector device 39. This device contains a narrow band bandpass 39a. In a modified embodiment the measurement radiation is coupled into the measurement cell via an optical system, thus without a glass fiber conduit, and in the same manner the detection radiation is coupled out of the measurement cell.

On side of the outlet an analysis stage 41 for quantitative analysis of the detector signal is connected with the detector device 39. The analysis stage 41 itself is on side of the outlet connected on the one hand with an inlet of the control signal of the dialysis apparatus 29 and on the other hand with a display unit 43, a storage unit 45 and a transmission device 47 which are designed for displaying, storage or transmission of the detection result or data derived herefrom in the analysis stage 41 to an external (central) processing unit.

FIGS. 3A and 3B respectively show schematic spectral diagrams for clarification of the operation of an embodiment of the invention for protein containing sample liquids which contain phenylalanine PHE, tyrosine TYR and tryptophan TRP. FIG. 3A depicts excitation wave length of these molecules, wherein an advantageous spectral range for excitation is marked with EXCITATION, and FIG. 3B depicts the intrinsic fluorescence radiation of the three molecules. Here it is marked an advantageous detection range DETECTION for the tryptophan molecule. It is obvious that the excitation wave length are around 280 nm, while the detection spectral range selected here is at 350 nm and a bit above. At this point the tryptophan molecule has a maximum in intrinsic fluorescence, while the intrinsic fluorescence spectra of phenylalanine and tyrosine exhibit no significant intensity in this spectral range. A corresponding detector or filter adjustment enables a high-selective detection of tryptophan.

The operation of the invention is not limited to the examples described herein and highlighted aspects, but is also possible in numerous modifications which are within the scope of skilled action. In particular other embodiments for a light source and a detector are possible, and are to be selected wisely depending on the specific excitation and emission wavelengths of molecules to be detected.

1-11. (canceled)

12. A detection device for quantitative detection of predetermined molecules, in particular medium-sized protein molecules, in a sample liquid with a light source for emitting measurement radiation, a section for irradiating the sample liquid and a light detector for measuring the detection radiation exiting the section for irradiating the sample liquid, as well as an analysis stage located downstream of the light detector for analyzing the detector signal, wherein the light source is designed such that the measurement radiation has a spectral range exciting the intrinsic fluorescence of a molecule to be determined, and the light detector is designed such that at least one spectral range of the detection radiation is measured, in which the molecule emits intrinsic fluorescence radiation.

13. The detection device according to claim 12, wherein the light source is a UV-radiation source emitting detection radiation exciting the intrinsic fluorescence of tryptophan, in particular at 280 nm, and the light detector is a photodiode or a photomultiplier, which is sensitive in a spectral range, in which intrinsic fluorescence radiation of tryptophan is emitted, in particular above 340 nm.

14. The detection device according to claim 12, wherein a narrowband filter is connected ahead of the light detector, which particularly defines a part of the spectral range between 340 nm and 400 nm as a detection range.

15. The detection device according to claim 12, wherein the light detector has a direction of arrival of light which is inclined with respect to a direction of emission of the light source.

16. The detection device according to claim 12, wherein an additional light detector for detecting the effect of the absorption of the sample liquid relating to the section for irradiating the sample liquid is arranged to the light source such that its direction of arrival of light coincides with the direction of emission of the light source.

17. The detection device according to claim 12 designed for continuous operation.

18. A dialysis apparatus having a detection device according to claim 12, wherein the section for irradiating the sample liquid is designed in the form of a section of a measurement cell or a liquid conduit which is passed through by spent dialysis fluid as the sample liquid being conveyed from the dialysis apparatus.

19. The dialysis apparatus according to claim 18, wherein a display unit which is connected with the analysis stage of the detection device is provided for displaying the analysis result or data derived from it and/or a storage device for storage of the detection result or data derived from it and/or a transmission device for transmission of the detection result or data derived from it to an external processing unit.

20. The dialysis apparatus according to claim 18, wherein the analysis stage of the detection device is connected with an input of a control device of the dialysis apparatus such that the operation of the dialysis apparatus is controllable in dependence on the detection result of the detection device.

21. The dialysis apparatus according to claim 19, wherein the analysis stage of the detection device is connected with an input of a control device of the dialysis apparatus such that the operation of the dialysis apparatus is controllable in dependence on the detection result of the detection device.

22. A method for quantitative detection of predetermined molecules, in particular medium size protein molecules, in a sample liquid, wherein the light source for emitting measurement radiation is run in a section for irradiating the sample liquid, and a light detector for measuring the detection radiation exiting the section for irradiating the sample liquid, as well as an analysis stage located downstream of the light detector are run for analyzing the detector signal, wherein the light source is carried out such that the measurement radiation has a spectral range exciting the intrinsic fluorescence of a molecule to be determined, and the light detector is carried out such that at least one spectral range of the detection radiation is measured, in which the molecule emits intrinsic fluorescence radiation.

23. The method according to claim 22 for observing the operation of a dialysis apparatus, wherein spent dialysis liquid passes through the section for irradiating the sample liquid as sample liquid.
